(19) United States
(12) Patent Application Publication
(10) Pub. No.: US 2008/0161680 A1
(43) Pub. Date: Jul. 3, 2008

(54) SYSTEM AND METHOD FOR SURGICAL NAVIGATION OF MOTION PRESERVATION PROSTHESIS

(75) Inventors: Ronald A. von Jako, Saugus, MA (US); Charles F. Lloyd, Reading, MA (US); Laurent J. Node-Langlois, Boston, MA (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/617,857

(22) Filed: Dec. 29, 2006

(51) Int. Cl. A61B 5/05; A61F 2/44; G06F 17/00

(52) U.S. Cl. 600/424; 600/407; 623/17.11; 702/94

(57) ABSTRACT

An image guided surgical system and method for targeting the precise patient specific anatomical placement of surgical instruments and motion preservation implants with surgical navigation. The system and method comprising a surgical navigation system; at least one imaging system coupled to the surgical navigation system; at least one computer coupled to the surgical navigation system and the imaging system having planning software for measuring clinical parameters of anatomy of a subject to be operated on; and at least one display for displaying imaging data, planning data and tracking data.

### BACKGROUND OF THE INVENTION

This disclosure relates generally to image-guided surgery (or surgical navigation). In particular, this disclosure relates to a planning system and method coupled with surgical navigation for the precise placement of motion preservation prostheses.

Medical navigation systems track the precise location of surgical instruments and implants in relation to multidimensional images of a patient's anatomy. Additionally, medical navigation systems use visualization tools to provide the surgeon with co-registered views of these surgical instruments and implants with the patient's anatomy. The multidimensional images of a patient's anatomy may include computed tomography (CT) imaging data, magnetic resonance (MR) imaging data, positron emission tomography (PET) imaging data, ultrasound imaging data, X-ray imaging data, or any other suitable imaging data, as well as any combinations thereof. Medical navigation technology has been applied to various areas of the body including the spinal column.

Persistent back pain is usually caused by degenerative disc disease. Normal spinal discs function as shock absorbers between the bones of the spine and as nerve protectors, allowing flexibility and movement. Degenerative disc disease results from discs that are damaged or degenerated, and have lost their form and function, leaving the nerves bare, and causing excruciating back pain. Degenerative disc disease may be caused by an unexpected injury or by aging, and includes any damage caused to one or more of the discs located between the vertebrae along the spine.

The modern trend for treating chronic degenerative disc disease is by total disc resection or replacement (TDR). Unlike spinal fusion, TDR is unique because it completely replaces the damaged disc and restores the disc space to its normal height and capacity without destroying the function of the joint. The prosthetic device used in TDR, consists of two end plates with a central core that is inserted between two vertebrae. This replaces the damaged disc and allows the surrounding vertebrae to remain intact. The implant encourages the vertebrae to grow into the prosthesis, enabling the joint to have a more normal range of motion, and diminishing stress on other discs. Surgical navigation helps to accomplish TDR through near real-time planning on saved X-ray images by virtual instruments superimposed over previously acquired X-ray images.

Current techniques for lumbar interbody placement devices are limited to CT, MR, X-ray fluoroscopy, templates, and simple marker devices such as a screw placed in the ventral midline of the vertebral body. Uses of these methods to diagnostically and surgically plan and determine the ideal position, orientation and alignment of implant devices are challenging and not always precisely accurate.

Specific devices such as motion preservation implants specifically artificial discs for TDR are challenging for the ideal placement and alignment and can lead to migration, disc subsidence, and decreased range of motion after surgery if not well orientated in the disc space.

Therefore, there is a need for a surgical navigation planning system and method for the ideal placement of motion preservation prostheses.

### BRIEF DESCRIPTION OF THE INVENTION

In an embodiment, a system for precise placement of a motion preservation prosthesis comprising a surgical navigation system; at least one imaging system coupled to the surgical navigation system; at least one computer coupled to the surgical navigation system and the imaging system having planning software for measuring clinical parameters of anatomy of a subject to be operated on; and at least one display for displaying imaging data, planning data and tracking data.

In an embodiment, a method for targeting the precise placement of surgical instruments and implants comprising performing pre-operative planning and imaging; providing surgical navigation with custom interfaces to surgical instruments and the implants to drive precise placement of the implants; and performing an intraoperative review of the placement of the implants.

In an embodiment, a computer-usable medium having computer readable instructions stored thereon for execution by a processor, the computer readable instructions comprising a planning routine for measuring clinical parameters of anatomy of a subject to be operated on and calculating patient specific dimensions for placement of an implant; a virtual template routine for providing a virtual template of surgical instruments and the implant; and a simulation routine for provides simulation of the range of motion of the anatomy of the subject being operated on.

In an embodiment, a computer program product for use with a computer, the computer program product comprising a computer-usable medium with computer readable instructions stored thereon for execution by a processor, the computer readable instructions stored thereon for execution by a processor performing a method comprising performing pre-operative imaging and planning; performing intraoperative navigation for the precise placement of an implant; and performing an intraoperative review to confirm the precise placement of the implant.

Various other features, objects, and advantages of the invention will be made apparent to those skilled in the art from the accompanying drawings and detailed description thereof.

### BRIEF DESCRIPTION OF THE DRAWINGS

FIG. 1 is an exemplary schematic diagram of an embodiment of an imaging and navigation system;

FIG. 2 is an exemplary block diagram of an embodiment of an imaging and navigation system;

FIG. 3 is an exemplary flow diagram of an embodiment of a method for precise positioning of an implant;

FIG. 4A is an exemplary diagram of an embodiment of a display illustrating various imaging views with planning software including a CAD implant model;

FIG. 4B is an exemplary diagram of an embodiment of a display illustrating various imaging views with planning software including a CAD implant model and simulation;

FIG. 5A is an exemplary diagram of an embodiment of a display illustrating various imaging views with surgical navigation software including a user interface;

FIG. 5B is an exemplary diagram of various embodiments of a plurality of instruments used in selection and positioning of an implant;

FIG. 6 is an exemplary diagram of a front view of a portion of anatomy illustrating placement of an artificial disc prosthesis; and

FIG. 7 is an exemplary flow diagram of an embodiment of a method for precise positioning of an implant.

### DETAILED DESCRIPTION OF THE INVENTION

In spinal surgical procedures, access to the body is obtained through one or more small percutaneous incisions or one larger incision. Surgical instruments and/or implants are inserted through these openings and directed to a region of interest within the body. Direction of the surgical instruments or implants through the body is facilitated by navigation technology wherein the real-time location of a surgical instrument or implant is measured and virtually superimposed on an image of the region of interest. The image may be a pre-acquired image, or an image obtained in near real-time or real-time using known imaging technologies such as computed tomography (CT), magnetic resonance (MR), positron emission tomography (PET), ultrasound, X-ray, or any other suitable imaging technology, as well as any combinations thereof.

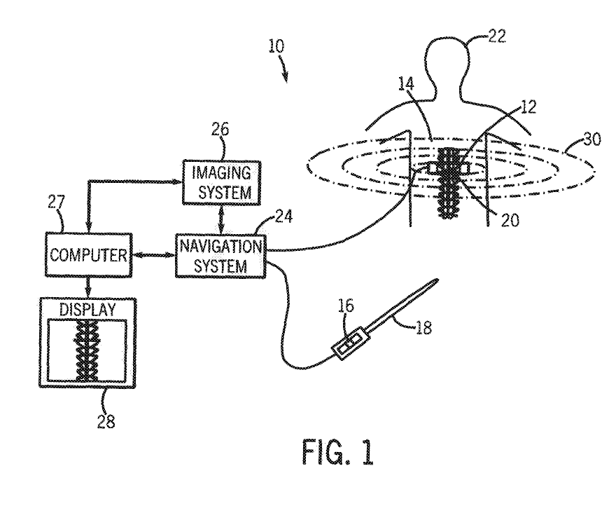

Referring now to the drawings, FIG. 1 is an exemplary schematic diagram of an embodiment of an imaging and navigation system. The system 10 includes at least one electromagnetic field generator 12 positioned proximate to a surgical field of interest 14, at least one electromagnetic sensor 16 attached to at least one navigated surgical instrument 18 to which an implant may be attached, the at least one electromagnetic sensor 16 communicating with and receiving data from the at least one electromagnetic field generator 12, a navigation system 24 coupled to and receiving data from the at least one electromagnetic sensor 16 and the at least one electromagnetic field generator 12, an imaging system 26 coupled to the navigation system 24 for performing imaging on a patient 22 in the surgical field of interest 14, a computer 27 coupled to the navigation system 24 and the imaging system 26, and a display 28 coupled to the computer 27 for displaying imaging and tracking data from the imaging system 26 and the navigation system 24.

In an exemplary embodiment, the at least one electromagnetic field generator 12 may be attached to a registration apparatus 20 that may be attached to the patient 22 in the surgical field of interest 14. The at least one electromagnetic field generator 12 creates a local reference frame for the navigation system 24 around the patient's anatomy.

The display 28 may be configured to show the real-time position and orientation of a model of the at least one surgical instrument 18 or at least one implant attached to the tip or end of the at least one surgical instrument 18 on a registered image of the patient's anatomy. The model of the at least one surgical instrument 18 or at least one implant may appear as a line rendering, a few simply shaded geometric primitives, or a realistic 3D model from a computer-aided design (CAD) file.

In an exemplary embodiment, the imaging system 26 and the navigation system 24 may be integrated into a single integrated imaging and navigation system with integrated instrumentation and software.

The system 10 enables a surgeon to continually track the position and orientation of the surgical instrument 18 or an implant attached to the surgical instrument 18 during surgery. An electromagnetic field 30 is generated around the at least one electromagnetic field generator 12. The at least one electromagnetic sensor 16 detects the electromagnetic field 30 generated by the at least one electromagnetic field generator 12 attached to the registration apparatus 20. The at least one electromagnetic sensor 16 may be an electromagnetic field receiver. The electromagnetic field receiver may be a receiver array including at least one coil or at least one coil pair and electronics for digitizing magnetic field measurements detected by the receiver array. The at least one electromagnetic field generator 12 may be an electromagnetic field transmitter. The electromagnetic field transmitter may be a transmitter array including at least one coil or at least one coil pair. It should, however, be appreciated that according to alternate embodiments the registration apparatus 20 may include at least one electromagnetic field receiver attached thereto and the surgical instrument 18 may include at least one electromagnetic field transmitter attached thereto.

The magnetic field measurements can be used to calculate the position and orientation of the surgical instrument 18 or an implant according to any suitable method or system. After the magnetic field measurements are digitized using electronics, the digitized signals are transmitted from the at least one electromagnetic sensor 16 to the navigation system 24. The digitized signals may be transmitted from the at least one electromagnetic sensor 16 to the navigation system 24 using wired or wireless communication protocols and interfaces. The digitized signals received by the navigation system 24 represent magnetic field information detected by the at least one electromagnetic sensor 16. The digitized signals are used to calculate position and orientation information of the surgical instrument 18 or implant. The position and orientation information is used to register the location of the surgical instrument 18 or implant to acquired imaging data from the imaging system 26. The position and orientation data is visualized on the display 28, showing in real-time the location of the surgical instrument 18 or implant on pre-acquired or real-time images from the imaging system 26. The acquired imaging data from the imaging system 26 may include CT imaging data, MR imaging data, PET imaging data, ultrasound imaging data, X-ray imaging data, or any other suitable imaging data, as well as any combinations thereof. In addition to the acquired imaging data from various modalities, real-time imaging data from various real-time imaging modalities may also be available.

The navigation system 24 is illustrated conceptually and may be implemented using any combination of dedicated hardware boards, digital signal processors, field programmable gate arrays, and processors. Alternatively, the navigation system 24 may be implemented using an off-the-shelf computer with a single processor or multiple processors, with the functional operations distributed between processors. As an example, it may be desirable to have a dedicated processor for position and orientation calculations as well as a processor for visualization operations. The navigation system 24 may be an electromagnetic navigation system utilizing electromagnetic navigation technology. However, other tracking or navigation technologies may be used.

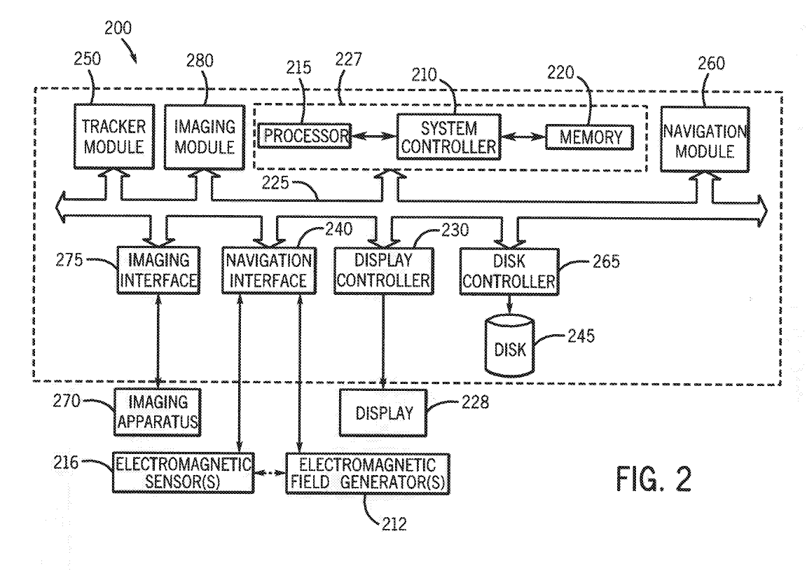

FIG. 2 is an exemplary block diagram of an embodiment of an imaging and navigation system 200. The imaging and navigation system 200 is illustrated conceptually as a collection of modules, but may be implemented using any combination of dedicated hardware boards, digital signal processors, field programmable gate arrays, and processors. Alternatively, the modules may be implemented using an off-the-shelf computer with a single processor or multiple processors, with the functional operations distributed between the processors. As an example, it may be desirable to have a dedicated processor for position and orientation calculations as well as a dedicated processor for visualization operations. As a further option, the modules may be implemented using a hybrid configuration in which certain modular functions are performed using dedicated hardware, while the remaining modular functions are performed using an off-the-shelf computer. In the embodiment shown in FIG. 2, the system includes a single computer 227 having a processor 215, a system controller 210 and memory 220. The operations of the modules may be controlled by the system controller 210.

The imaging and navigation system 200 includes at least one electromagnetic field generator 212 that is coupled to a navigation interface 240. The at least one electromagnetic field generator 212 generates at least one electromagnetic field that is detected by at least one electromagnetic field sensor 216. The navigation interface 240 receives digitized signals from at least one electromagnetic sensor 216. The navigation interface 240 includes at least one Ethernet port. The at least one Ethernet port may be provided, for example, with an Ethernet network interface card or adapter. However, according to various alternate embodiments, the digitized signals may be transmitted from the at least one electromagnetic sensor 216 to the navigation interface 240 using alternative wired or wireless communication protocols and interfaces.

The digitized signals received by the navigation interface 240 represent magnetic field information from the at least one electromagnetic field generator 212 detected by the at least one electromagnetic sensor 216. In the embodiment illustrated in FIG. 2, the navigation interface 240 transmits the digitized signals to a tracker module 250 over a local interface 215. The tracker module 250 calculates position and orientation information based on the received digitized signals. This position and orientation information provides a location of a surgical instrument or implant.

The tracker module 250 communicates the position and orientation information to a navigation module 260 over a local interface 215. As an example, this local interface 215 is a Peripheral Component Interconnect (PCI) bus. However, according to various alternate embodiments, equivalent bus technologies may be substituted.

Upon receiving the position and orientation information, the navigation module 260 is used to register the location of the surgical instrument or implant to acquired patient data. In the embodiment illustrated in FIG. 2, the acquired patient data is stored on a disk 245. The acquired patient data may include computed tomography data, magnetic resonance data, positron emission tomography data, ultrasound data, X-ray data, or any other suitable data, as well as any combinations thereof. By way of example only, the disk 245 is a hard disk drive, but other suitable storage devices may be used.

The acquired patient data is loaded into memory 220 from the disk 245. The acquired patient data is retrieved from the disk 245 by a disk controller 265. The navigation module 260 reads from memory 220 the acquired patient data. The navigation module 260 registers the location of the surgical instrument or implant to acquired patient data, and generates image data suitable to visualize the patient image data and a representation of the surgical instrument or implant. The image data is transmitted to a display controller 230 over a local interface 215. The display controller 230 is used to output the image data to display 228.

In another exemplary embodiment, the imaging and navigation system 200 may include an imaging apparatus 270 coupled to an imaging interface 275 for receiving real-time imaging data. The imaging data is processed in an imaging module 280. The imaging apparatus 270 provides the ability to display real-time position and orientation information of a surgical instrument or implant on the display 228.

While one display 228 is illustrated in the embodiment in FIG. 2, alternate embodiments may include various display configurations. Various display configurations may be used to improve operating room ergonomics, display different views, or display information to personnel at various locations.

In an exemplary embodiment, the at least one electromagnetic sensor or the at least one electromagnetic field generator may be a microsensor, microcoil, or microarray that may be removably attached to a bone in the surgical field of interest of a patient to be operated on. The at least one microsensor allows a surgeon to more accurately place prostheses during surgery. The at least one microsensor enables a surgeon to continually track the position and orientation of the anatomy of interest and implants during surgery.

The at least one microsensor may be an electromagnetic field generator that includes microcoils for generating a magnetic field, and the at least one electromagnetic sensor may be an electromagnetic field receiver. An electromagnetic field is generated around the at least one microsensor. The at least one electromagnetic sensor is brought into proximity with the at least one microsensor to receive magnetic field measurements from the at least one microsensor for calculating the position and orientation of the at least one microsensor. The at least one electromagnetic sensor receives tracking data from the at least one microsensor that may measure in real-time the range of motion of the anatomy of interest. The electromagnetic field receiver may be a receiver array including at least one coil or at least one coil pair and electronics for digitizing magnetic field measurements detected by the receiver array. It should, however, be appreciated that according to alternate embodiments the at least one microsensor may be an electromagnetic field receiver and the at least one electromagnetic sensor may be an electromagnetic field generator.

The at least one microsensor is configured to provide range of motion information and for tracking the tip of a surgical instrument or implant. The at least one microsensor is part of the navigation system used to track movement of the anatomy being replaced and the implants. The navigation system is configured to calculate the relative locations of the microsensor based on the received digitized signals. The navigation system further registers the location of the microsensor to the acquired imaging data, and generates imaging data suitable to visualize the image data and representations of the microsensor. The at least one microsensor may be used to measure biomechanical parameters of the anatomy of interest. These biomechanical parameters allow a surgeon to implant a prosthesis by taking into account the size, shape and movement of the anatomy being replaced. The at least one microsensors may be passively powered, powered by an external power source, or powered by an internal battery.

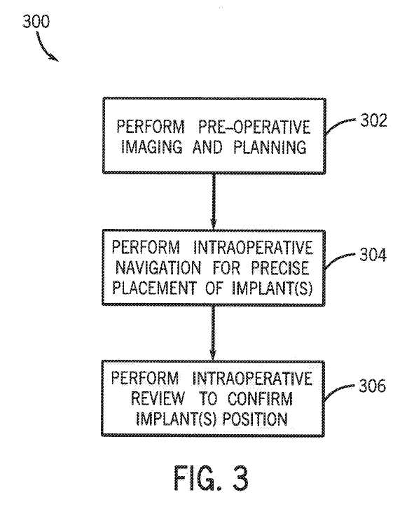

FIG. 3 is an exemplary flow diagram of an embodiment of a method 300 for precise positioning of an implant. This method is accomplished with the use of a surgical navigation system, planning software, and at least one imaging system. The surgical navigation system with the addition of specific surgical navigation planning software coupled with key instruments in the placement of motion preservation implant devices, the surgeon will preoperatively and intraoperatively be able to plan and guide implant devices to patient specific anatomical dimensions and alignments with less need for estimations, X-ray and with greater confidence and speed. The surgical navigation system provides the custom interfaces to the surgical instruments that drive the precise placement of various implants, such as vertebral interbody devices, for both lumbar and cervical spine as well as the thoracic spine, as necessary.

The method includes performing preoperative imaging and planning prior to an implant surgery 302, performing intraoperative navigation for the precise placement of an implant 304, and performing an immediate intraoperative review to confirm the implant position 306. Use of preoperative and intraoperative surgical navigation software may assist in the planning and placement of implants based on patient specific anatomy to improve the longevity of a motion preservation implant.

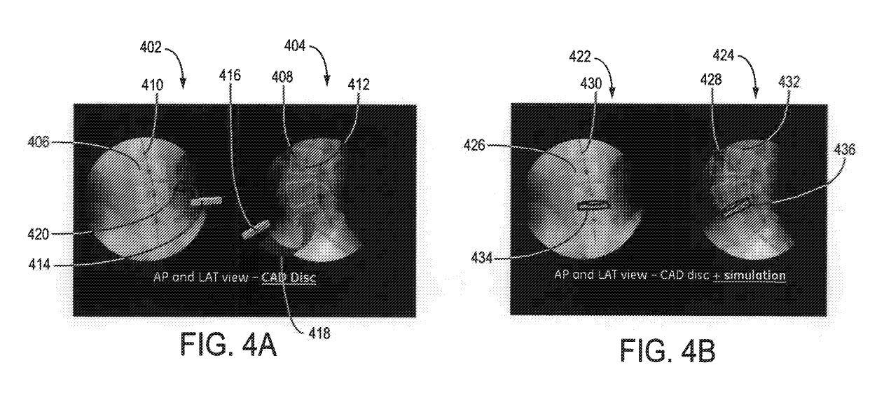

FIGS. 4A, 4B, 5A, 5B, and 6 are all provided to illustrate an example TDR procedure utilizing an embodiment of the system and method of the invention. An artificial disc includes two endplates and a central core situated between the two endplates. The endplates are available in different sizes and configurations. The cores are available in different sizes (depth and height) to fit the various endplates.

FIG. 4A is an exemplary diagram of an embodiment of a display illustrating anterior-posterior (AP) 402 and lateral (LAT) 404 views of an image of a portion of a patient's spine 406, 408 including a CAD model of an artificial disc prosthesis 414, 416. FIG. 4A illustrates the use of planning software on a surgical navigation system for performing a TDR procedure. The planning software includes methods for calculating patient specific dimensions, ideal instrument and implant alignments, instrument guidance and intraoperative range of motion calculations for accessing long term kinematic profiles of TDR prosthesis. For a TDR procedure, the patient specific dimensions may include current disc height, vertebral body height, endplate length and width.

FIG. 4B is an exemplary diagram of an embodiment of a display illustrating AP 422 and LAT 424 views of an image of a portion of a patient's spine 426, 428 including a CAD model of an artificial disc prosthesis 434, 436 and simulation of the CAD model of the artificial disc prosthesis 434, 436 being inserted into a disc space of the spine. FIG. 4B illustrates the use of simulation software on a surgical navigation system for performing a TDR procedure. The simulation software is used to simulate spine mobility during surgery and compare it with the preoperative diagnostics.

For example, the images shown in FIGS. 4A and 4B may be obtained using CT, MR, PET, ultrasound, X-ray or any suitable imaging technology, as well as any combinations thereof. The AP 402, 422 view is used to define the axial midline 410, 430 of the vertebral body, which is critical to proper placement of an implant. The LAT 404, 424 view is used to define the sagittal midline 412, 432 of the vertebral body. The planning software will assist the surgeon to identify and mark the critical axial midline 410, 430 in the AP 402, 422 view and the sagittal midline 412, 432 in the LAT 404, 424 view for each and all vertebral disc levels to be operated on.

In addition to help in verifying the ideal axial and sagittal midlines and depth for an implant, the planning software also helps in selecting the correct implant type and size. As illustrated above, this is accomplished by providing a virtual template of an implant, a 3D model of the implant, and simulation for positioning the virtual implant template off of the axial midline and sagittal midline measurements. The planning software selects the best implant template according to the patient's anatomy. Following this, the planning software selects the ideal instrument and implant alignments for navigation of the instruments and implant. Knowing the geometry of the implant and its mechanical behavior, the planning software will be able to simulate the range of motion of the patient, and simulate the placement of the implant. This simulation information is combined with the information contained in the dynamic images.

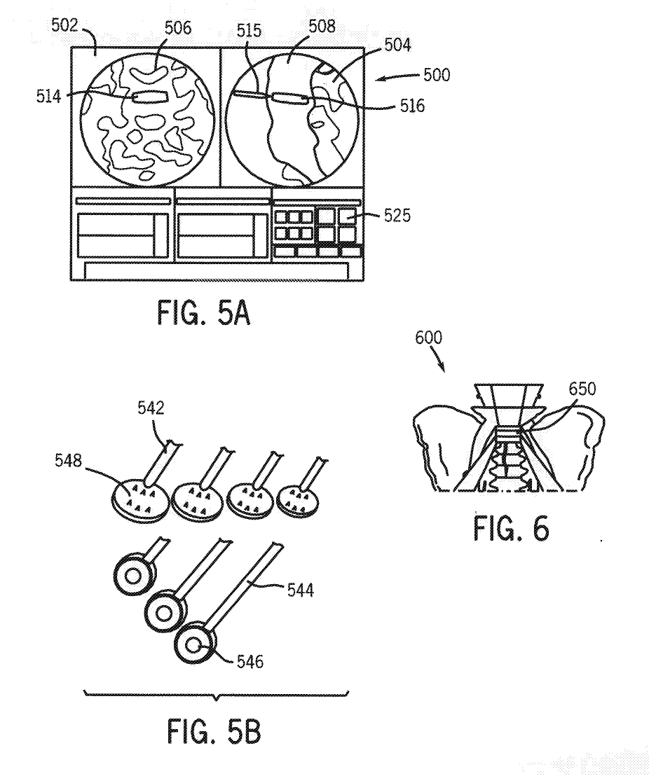

FIG. 5A is an exemplary diagram of an embodiment of a display 500 illustrating AP 502 and LAT 504 views of an image of a portion of a patient's spine 506, 508 including surgical navigation models 514, 515, 516 and a user interface 525. FIG. 5A illustrates the use of surgical navigation during the TDR procedure. The surgical navigation display 500 includes the interface and computer navigation of bone markers, trial and/or sizing gauges used to measure the superior and inferior vertebral endplates and their lateral borders or “endplate mapping” for ovoid-shape vertebral bones, sacral level-one (S1) slope planning (for an angled disc space), depth gauges (solid and virtual models), disc space preparation and cleaning instruments such as navigated osteomes to shave the endplates, curettes, rongeurs, kerissons, debrieders, a chisel (for precise midline cut to place implant in upper and lower vertebral body ends) and shavers for osteophytes (bone spurs), spreaders for controlled distraction and the insertion instruments for vertebral endplates and implant pinion core.

The user interface 525 provides the interface for surgical navigation. It may include the selection of different views of the planning, pre-operative imaging, navigation, and intraoperative imaging displays for placement and final review of the implant position and orientation. The features and orientation of each view may be customized using the interface 525. Using pre-operative information like dynamic images combined to the final position of the implant, the system will be able to simulate the mobility of the patient (around the specific instrumented segment). A real-time imaging axial view may also help to check the gap between articular facets.

FIG. 5B is an exemplary diagram of various embodiments of a plurality of instruments 542, 544, 546, 548 used in selection and positioning of an implant. For a TDR procedure, the instruments 500 may include sizing gauges 548, trial insertion guides, and pilot drivers 542 for endplates; and trial cores 546, spreading and insertion forceps, and insertion instruments 544 for the core. These instruments 542, 544, 546, 548 are all tracked using the surgical navigation system, and are used to determine endplate size and position, center of disc space, height and depth of disc space, and ideal core height and depth.

Matched with the software calculations done with both surface and non-surface matching methods for bone registration, a 2D vertebral X-ray image in any view can be used to display the measurements of the segmental bones for height, width, depth as well as disc height, that can also be updated with distraction and axial load patterns with the proper released disc to assist in the avoidance of possible injury to the posterior elements of the spine (posterior longitudinal ligament and annulus). With 3D CT volumetric multi-planar reconstructions (MPR), highly precise measurements can be created patient specifically when placing a TDR, an interbody fusion (IBF) cage, or for nucleus replacement devices. Tracking both vertebral bodies would better insure the balance and side-to-side space balance (symmetry) to help avoid disc migration from one side versus the opposite. A virtual template is superimposed with measurements over the different imaging views and the live components can be computer navigated in near real-time as virtual instruments and implant components to be aligned over the predefined anatomical templates for precise orientations and alignments.

FIG. 6 is an exemplary diagram 600 of a front view of a portion of anatomy illustrating placement of an artificial disc prosthesis 650. A final step in the process is an immediate intraoperative review of the implant position, using for example a 3D fluoroscopic X-ray imaging system, to confirm the implant position before closing the surgical incision. The position and orientation of the implant is compared to the original disc in pre-operative planning, and the ideal position and orientation determined during measurements and calculations determined during the navigated surgical procedure. This review image may be based on a 3D live image such as from an intraoperative CT scanner or a 3D fluoroscopic acquisition system. The goal is to check, compare and save the implant position before closure of the incision.

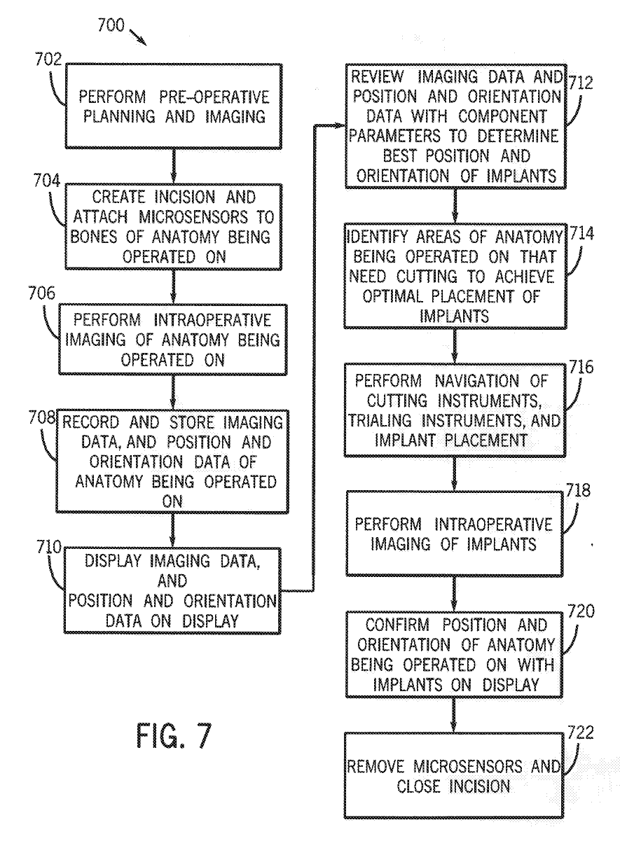

FIG. 7 is an exemplary flow diagram of an embodiment of a method 700 for precise positioning of an implant. The method 700 includes performing pre-operative planning and imaging 702. Planning software measures with accuracy any clinical anatomy parameters needed for implant placement. A next step is creating an incision and attaching microsensors to bones of anatomy being operated on 704. At least one microsensor (microcoil, microarray, etc.) may be removably attached to the bones for tracking range of motion of anatomy and navigate placement of the implant. A next step includes performing intraoperative imaging of anatomy being operated on 706. An imaging system is used to provide parametric models and the like as virtual jigs for measuring the morphometric spinal segments for the process of registration and navigating implant placement and implant components into their ideal alignments for any vertebral interbody space. A next step includes recording and storing imaging data, and tracking (position and orientation) data of the anatomy being operated on 708. All the clinical information relative to the patient during the surgical workflow is recorded. The method further includes displaying imaging data and tracking (position and orientation) data of the anatomy being operated on, on a display 710. The method further includes reviewing imaging data and tracking (position and orientation) data of the anatomy being operated on with component parameters to determine the best position and orientation of the implant 712. Another step in the process includes identifying areas of the anatomy being operated on that need cutting to achieve optimal placement of the implant 714. The navigation system allows a surgeon to navigate the use of surgical instruments and the placement of the implant 716, including performing trialing of the implant 716. The method further includes performing intraoperative imaging of the implant 718. The surgeon can then confirm the position and orientation of the implant with the position and orientation of the original anatomy being operated on, on a display 720. This includes an immediate intraoperative review of the implant position to confirm the implant position before closing the surgical incision. The final step is removing the microsensors and closing the incision 722.

In an embodiment of a method for performing a TDR, a surgeon performs pre-operative planning and imaging. Planning software is used to track placement of implants, including insertion of artificial disc prosthesis or an interbody cage. The planning software creates a virtual template of the implant and provides simulation of placement of the implant. The system may include adding at least one microsensor to the vertebral bodies for tracking movement of the vertebral bodies. Imaging is performed to estimate endplate size and angle. An incision is made. A discectomy is performed to remove damaged or diseased disc. Instrumentation is used to hold disc space open. Endplate preparation is performed. Planning software is used to create template of surface of each bone end plate. May need to shave bone to create flat surfaces for endplates. Markers are placed to help determine axial midline, sagittal midline, and disc space depth and width to create template for disc. Simulation and trialing is performed to determine correct type, size, and position for endplates and core, and range of motion. Planning software takes into account patient and implant range of motion, implant dimensions, and patient anatomy. Verify correct placement of trial. Navigation is used during trial placement and to place disc prosthesis, including endplates and core into disc space. Plan and track ideal midline and depth placement of two endplates. Plan and track ideal transverse and dorsal depth position for core. The planning software may include S1 slope planning software, bone morphing endplate software, and virtual template software. The endplates may need to be non-parallel to accommodate the natural curve of spine. The final position of artificial disc prosthesis is confirmed and verified using an imaging system. This includes an immediate intraoperative review of the implant position to confirm the implant position before closing the surgical incision. The incision is closed.

The embodiments described above include several advantages. For example, the system coupled with surgical navigation and imaging will assist a surgeon in overcoming moments of X-ray parallax and improved range of motion that may result in improved clinical outcomes and lower the incidence of adjacent segment degenerative disc disease. In the cervical spine coronal and sagittal alignments will be calculated with computer assisted precision and the dependence on X-ray and naked eye estimations at any vertebral level alone will be significantly improved. The surgical team will be less dependent on bone screws as X-ray markers that are sometimes difficult to place as well as retrieve. In addition, there may be less dependency on anatomical landmarks from X-ray fluoroscopy, such as the spinous process and pedicles that may not always be anatomically aligned due to deformity, degeneration, previous surgery, or trauma. The surgical team will also have greater confidence, steer the surgical disc preparation instruments and implant components to the proper anterior midline, visualize bi-lateral border equal distances, perform “endplate mapping” for level and flat surfaces (important in cervical spine) individually measured for both lordosis and kyphosis, use a virtual positive stop mechanism for the navigated insertion instruments, perform sacral level one (L5-S1) slope planning, and calculate sagittal positions for depth and alignments. Planning the ideal transverse and dorsal depth positions for final placement will be assisted by the surgical navigation software and guided by surgical navigation for the prosthetic ideal core height and depth placement by individual or dual simultaneous vertebral endplate tracking. The system and method will also improve surgical confidence in implant placements, minimize fluoroscopy time and X-ray exposure, minimize the multiple manipulations of mobile imaging system use leading to less chance of cross contamination in the sterile field, minimize the overall intra-operative time, act as an enabler for least invasive approaches that decrease morbidity and improve the postoperative outcomes.

The system and method is a unique solution benefiting the patient and operating surgeons through the adoption of surgical navigation and imaging with sophisticated algorithms for measurement calculations for bone specific dimensions and instruments to provide the precise placement of an implant.

It should be appreciated that according to alternate embodiments, the at least one electromagnetic sensor or microsensor may be an electromagnetic receiver, an electromagnetic generator (transmitter), or any combination thereof. Likewise, it should be appreciated that according to alternate embodiments, the at least one electromagnetic field generator may be an electromagnetic receiver, an electromagnetic transmitter or any combination of an electromagnetic field generator (transmitter) and an electromagnetic receiver.

Several embodiments are described above with reference to drawings. These drawings illustrate certain details of specific embodiments that implement the systems, methods and programs of the invention. However, the drawings should not be construed as imposing on the invention any limitations associated with features shown in the drawings. This disclosure contemplates methods, systems and program products on any machine-readable media for accomplishing its operations. As noted above, the embodiments of the may be implemented using an existing computer processor, or by a special purpose computer processor incorporated for this or another purpose or by a hardwired system.

As noted above, embodiments within the scope of the included program products comprising machine-readable media for carrying or having machine-executable instructions or data structures stored thereon. Such machine-readable media can be any available media that can be accessed by a general purpose or special purpose computer or other machine with a processor. By way of example, such machine-readable media may comprise RAM, ROM, PROM, EPROM, EEPROM, Flash, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such a connection is properly termed a machine-readable medium. Combinations of the above are also included within the scope of machine-readable media. Machine-executable instructions comprise, for example, instructions and data which cause a general purpose computer, special purpose computer, or special purpose processing machines to perform a certain function or group of functions.

Embodiments are described in the general context of method steps which may be implemented in one embodiment by a program product including machine-executable instructions, such as program code, for example in the form of program modules executed by machines in networked environments. Generally, program modules include routines, programs, objects, components, data structures, etc. that perform particular tasks or implement particular abstract data types. Machine-executable instructions, associated data structures, and program modules represent examples of program code for executing steps of the methods disclosed herein. The particular sequence of such executable instructions or associated data structures represent examples of corresponding acts for implementing the functions described in such steps.

Embodiments may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of the invention might include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated machine-readable media provide nonvolatile storage of machine-executable instructions, data structures, program modules and other data for the computer.

The foregoing description of embodiments has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments and with various modifications as are suited to the particular use contemplated.

Those skilled in the art will appreciate that the embodiments disclosed herein may be applied to the formation of any medical navigation system. Certain features of the embodiments of the claimed subject matter have been illustrated as described herein, however, many modifications, substitutions, changes and equivalents will now occur to those skilled in the art. Additionally, while several functional blocks and relations between them have been described in detail, it is contemplated by those of skill in the art that several of the operations may be performed without the use of the others, or additional functions or relationships between functions may be established and still be in accordance with the claimed subject matter. It is, therefore, to be understood that the appended claims are intended to cover all such modifications and changes as fall within the true spirit of the embodiments of the claimed subject matter.

While the invention has been described with reference to several embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

What is claimed is:

1. A system for precise placement of a motion preservation prosthesis comprising:
a surgical navigation system;
at least one imaging system coupled to the surgical navigation system;
at least one computer coupled to the surgical navigation system and the imaging system having planning software for measuring clinical parameters of anatomy of a subject to be operated on; and
at least one display for displaying imaging data, planning data and tracking data.

2. The system of claim 1, wherein the planning software calculates patient specific dimensions for placement of the implant.

3. The system of claim 1, wherein the planning software is used to identify and mark the axial midline and sagittal midline on at least two views of images displayed on the at least one display for the anatomy being operated on.

4. The system of claim 1, wherein the planning software provides the best implant template according to the anatomy being operated on.

5. The system of claim 1, wherein the planning software and imaging provides simulation of the range of motion of the anatomy being operated on.

6. The system of claim 1, wherein the surgical navigation system provides navigation of bone markers, surgical instruments and implants.

7. The system of claim 1, further comprising a virtual template of the implant superimposed with measurements over different imaging views and providing surgical navigation of surgical instruments and implants.

8. The system of claim 1, wherein the at least one imaging system is a real-time fluoroscopic imaging system that provides updated images during surgery.

9. The system of claim 1, wherein the at least one imaging system is used to review, compare and save the implant position on the anatomy being operated on prior to closure of the surgical incision.

10. The system of claim 1, wherein the at least one display may include planning images, navigation images, and implant position images.

11. The system of claim 1, further comprising at least one microsensor attached to bones of the anatomy being operated on.

12. The system of claim 1, further comprising at least one microsensor attached to bones of the anatomy being operated on.

13. The system of claim 12, wherein the at least one microsensor is an electromagnetic field generator including at least one microcoil for generating an electromagnetic field.

14. The system of claim 12, wherein the at least one microsensor is an electromagnetic receiver for receiving an electromagnetic or magnetic field.

15. A method for targeting the precise placement of surgical instruments and implants comprising:
performing pre-operative planning and imaging;
providing surgical navigation with custom interfaces to surgical instruments and the implants to drive precise placement of the implants; and
performing an intraoperative review of the placement of the implants.

16. The method of claim 15, wherein the pre-operative planning includes planning software that calculates patient specific dimensions for placement of the implants.

17. The method of claim 16, wherein the planning software is used to identify and mark the axial midline and sagittal midline on at least two views of images displayed on the at least one display for the anatomy being operated on.

18. The method of claim 16, wherein the planning software and imaging provide simulation of the range of motion of the anatomy being operated on.

19. The method of claim 15, wherein the surgical navigation provides navigation of bone markers, surgical instruments and implants.

20. The method of claim 15, wherein the intraoperative review includes reviewing, comparing and saving the implant position on the anatomy being operated on prior to closure of the surgical incision.

21. A computer-usable medium having computer readable instructions stored thereon for execution by a processor, the computer readable instructions comprising:
a planning routine for measuring clinical parameters of anatomy of a subject to be operated on and calculating patient specific dimensions for placement of an implant;
a virtual template routine for providing a virtual template of surgical instruments and the implant; and
a simulation routine for provides simulation of the range of motion of the anatomy of the subject being operated on.

22. A computer program product for use with a computer, the computer program product comprising a computer-usable medium with computer readable instructions stored thereon for execution by a processor, the computer readable instructions stored thereon for execution by a processor performing a method comprising:
performing pre-operative imaging and planning;
performing intraoperative navigation for the precise placement of an implant; and
performing an intraoperative review to confirm the precise placement of the implant.
